(19) United States
(12) Patent Application Publication
(10) Pub. No.: US 2026/0306838 A1
(43) Pub. Date: Oct. 8, 2026

(54) IMPLANTABLE DEVICE AND METHOD FOR TREATING GASTRO ESOPHAGEAL REFLUX DISEASE (GERD) AND THE DIGESTIVE SYSTEM

(71) Applicant: GERDCARE MEDICAL LTD., Yokneam Illit (IL)

(72) Inventors: David HANUKA, Haifa (IL); Mordechay ESH, Givatayim (IL); Giora ARBEL, Hod Hasharon (IL); Meir OR, Kfar Eshchar (IL)

(21) Appl. No.: 19/481,644

(22) PCT Filed: May 7, 2024
(86) PCT No.: PCT/IB2024/054440

§ 371 Date: Nov. 5, 2025

Related U.S. Application Data
(60) Provisional application No. 63/464,577, filed on May 7, 2023.

(51) Int. Cl. A61N 1/36; A61N 1/05; A61N 1/08

(52) U.S. Cl. CPC A61N 1/36007; A61N 1/0504; A61N 1/08

(57) ABSTRACT

The subject matter discloses a method and apparatus for treating digestive system disorders, the method including: subcutaneously implanting in a patient's abdomen a pulse generator and plurality of electrodes, the electrodes are in electrical contact with target regions in the patient's abdomen; and delivering electrical stimulation to activate said target regions

### FIELD OF THE INVENTION

The present disclosure relates to medical devices in general, and to medical devices related to digestive system and GERD in particular.

### BACKGROUND OF THE INVENTION

Amongst other digestive system diseases, Gastro Esophageal Reflux Disease (GERD) is the most common outpatient gastroenterological diagnosis in the US, with a prevalence rate of 10% 30% and an annual incidence of 0.38%-0.45% in the western world. In the US, 20% of the adult population experience GERD-related symptoms weekly and 7% daily. GERD is known to significantly reduce health-related quality of life and to result in a marked economic burden on the healthcare system.

GERD is caused by undesired movement of stomach acidic content into the esophagus. Such movement is typically caused by changes in the barrier between the stomach and the esophagus, including abnormal relaxation of the lower esophageal sphincter (LES), impaired expulsion of gastric reflux from the esophagus, or hiatal hernia. Amongst others, GERD symptoms may include abdominal pain, heartburn, regurgitation, chest pain, soar taste and asthma. GERD complications may include mucosal damage, erosive esophagitis (EE), Barret disease and esophageal cancer.

Typical GERD treatments include lifestyle changes, medications such as proton pump inhibitors (PPI), H2 receptor blockers or antacids, and surgery.

Although the effectiveness of current GERD treatments is generally accepted, unmet needs and significant challenges remain. Approximately 10%-15% of adult patients with EE fail to achieve complete healing after 8 weeks of treatment. Moreover, even when continuing the initial healing dose, 15%-23% of adult EE patients with Los Angeles grades A and B, and 24%-41% with grades C and D, relapse within 6 months while on treatment. Further, up to 40 % of non-erosive reflux disease (NERD) adult patients remain symptomatic while on standard dose (once daily) PPI therapy.

Concerns are raised regarding the safety of prolonged use of PPI drugs. These concerns are associated with various possible adverse effects such as osteoporosis, kidney diseases, neurological disorders, dementia, liver diseases, anemia and others.

Surgical GERD therapy typically includes the fundoplication technique, which can be applied via open and endoscopic interventions. Fundoplication techniques provide an alternative to patients who do not respond to medication therapy, or are reluctant to use such medications for long periods of time. In recent years, various endoscopic intervention methods were developed and are used to treat GERD by minimally invasive methods. Yet, debatable effectiveness along with potential complications have limited their use in clinical practice.

U.S. Pat. No. 10,426,955 discloses methods for implanting electrodes and treating a patient with gastroesophageal reflux disease. It discloses a device and methodologies for the treatment of transient lower esophageal sphincter relaxations (tLESRs).

U.S. Pat. No. 7,660,636 discloses an electrical stimulation device and method for the treatment of dysphagia. In some embodiment, the electrical stimulation device includes one or more channels of electrodes each of which includes a first electrode positioned in electrical contact with tissue of a target region of a patient and a second electrode positioned in electrical contact with tissue of a posterior neck region or a posterior thoracic region of the patient. A series of electrical pulses are then applied to the patient through the one or more channels of electrodes in accordance with a procedure for treating dysphagia.

U.S. Pat. No. 5,716,385 discloses an electronic pacemaker used to counter-act crural diaphragm relaxation thereby preventing and/or treating gastroesophageal reflux. The pacemaker can be implantable, or be connected to the skeletal muscles of the crural diaphragm through the skin. A sensor is used to identify spontaneous intermittent relaxations of the diaphragm. During these spontaneous intermittent relaxations, one or more electrodes are used to stimulate the skeletal muscles of the crural diaphragm to cause contraction of the lower esophageal sphincter.

### SUMMARY OF THE INVENTION

According to an aspect of some embodiments of the present invention, there is provided a method for treating patients with digestive system disorders, comprising subcutaneously implanting in a patient's abdomen a pulse generator and a plurality of electrodes, the electrodes being in electrical contact with target regions in the patient's abdomen; and delivering electrical stimulation to activate the target regions.

According to some embodiments of the invention, the target regions include muscles, nerves, muscle-nerve junctions, or any combination thereof.

According to some embodiments of the invention, the electrical stimulation is optimized for manipulating the patient's digestive system in a manner that will lead to alteration in a rate of ingesta flow in a desired direction.

According to some embodiments of the invention, the electrical stimulation includes a series of current-controlled asymmetrical bursts intended to generate asymmetrical contractions of selected abdominal muscles; wherein a direction of a fast phase of the bursts being upward and a direction of a slow phase of the bursts being downward.

According to some embodiments of the invention, the alteration is a reduction of ingesta backflow from the patient's stomach to the esophagus, to alleviate GERD symptoms.

According to some embodiments of the invention, the alteration is an increasing of ingesta flow from the patient's stomach to the duodenum, to alleviate gastroparesis symptoms.

According to some embodiments of the invention, the alteration is an increasing of ingesta flow from the patient's colon to the rectum, to alleviate constipation symptoms.

According to some embodiments of the invention, the electrical stimulation includes a series of current-controlled asymmetrical bursts intended to generate asymmetrical contractions of selected abdominal muscles; wherein a direction of a fast phase of the bursts being upward and a direction of a slow phase of the bursts being downward.

According to some embodiments of the invention, the method further comprises implanting at least one sensor, wherein the stimulation is synchronized with at least one bodily signal acquired by the at least one sensor.

According to some embodiments of the invention, the sensor comprises at least one member of: accelerometer, gyroscope, magnetic compass, inclinometer, piezoelectric sensor, electrocardiogram (ECG), electroencephalogram (EEG), electromyograph (EMG), microphone, electric impedance sensor, oximeter, optical interferometer, pH meter, and opto-sensor.

According to some embodiments of the invention, the bodily signal comprises at least one member of: breathing, heart beating, blood pressure, muscular or ligamentous tension, body posture, arousal condition, or muscle activity level.

According to some embodiments of the invention, the activation of target regions is adapted for invoking a bodily reflex.

According to some embodiments of the invention, the reflex comprises at least one member of straining esophageal reflex and straining crural reflex.

According to an aspect of some embodiments of the present invention, there is provided a subcutaneously implantable device for treating digestive system disorders, comprising a control unit; a power source; a pulse generator; and a plurality of electrodes adapted to be placed in electrical contact with the abdominal target regions; wherein the pulse generator is designed to deliver stimulation signals via the electrodes to activate the target regions.

According to some embodiments of the invention, the electrical stimulation is characterized by maximum voltage of +20V, maximum current of 20 mA, pulse frequency range of 20 Hz-50 Hz, burst frequency range of 0.2 Hz-4 Hz and pulse width of 25 μs-300 μs.

According to some embodiments of the invention, the digestive system disorders are at least one member of a group consisting gastroesophageal reflux disease (GERD), gastroparesis, obesity, incontinence and constipation.

According to some embodiments of the invention, the electrical stimulation includes repetitive pulse bursts, and a shape of the burst is set in accordance with a desired physiological effect.

According to some embodiments of the invention, the implantable device further comprises at least one sensor.

According to some embodiments of the invention, the electrical stimulation is synchronized with at least one bodily signal acquired by the sensor.

According to some embodiments of the invention, the sensor comprises at least one member of: accelerometer, gyroscope, magnetic compass, inclinometer, piezoelectric sensor, electrocardiogram (ECG), electroencephalogram (EEG), electromyograph (EMG), microphone, electric impedance sensor, oximeter, optical interferometer, pH meter, and opto-sensor.

According to some embodiments of the invention, the bodily signal comprises at least one member of: breathing, heart beating, blood pressure, muscular or ligamentous tension, body posture, arousal condition, or muscle activity level.

Unless otherwise defined, all technical and/or scientific terms used herein have the same meaning as commonly understood by one of ordinary skills in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWING

The present disclosed subject matter will be understood and appreciated more fully from the following detailed description taken in conjunction with the drawings in which corresponding or like numerals or characters indicate corresponding or like components. Unless indicated otherwise, the drawings provide exemplary embodiments or aspects of the disclosure and do not limit the scope of the disclosure.

In the drawings:

FIG. 1A schematically illustrates a device for treating patients suffering from digestive system disorders, in accordance with some exemplary embodiments of the disclosed subject matter;

FIG. 1B schematically illustrates a partially sectioned view of the shell of FIG. 1A, in accordance with some exemplary embodiments of the disclosed subject matter;

FIG. 2 shows an exemplary device for treating digestive system disorders as implanted in a patient's abdomen, in accordance with some exemplary embodiments of the disclosed subject matter;

FIGS. 3A and 3B show waveforms of exemplary electrical stimulations comprising asymmetrical bursts, in accordance with some exemplary embodiments of the disclosed subject matter;

FIG. 3C shows waveforms of an exemplary electrical stimulation comprising symmetrical bursts, in accordance with some exemplary embodiments of the disclosed subject matter;

FIG. 3D shows a detailed view of a burst of any one of FIGS. 3A-3C, in accordance with some exemplary embodiments of the disclosed subject matter; and

FIG. 4 schematically illustrates a portion of exemplary data acquired by a sensor in accordance with some embodiments of the disclosed subject matter.

### DETAILED DESCRIPTION

Embodiments of the present invention disclose a method for treating patients suffering from digestive system disorders, and in particular gastroesophageal reflux disease (GERD), gastroparesis, constipation, incontinence and obeism. The method includes implanting a pulse generator and a plurality of electrodes in a subcutaneous location and delivering electrical stimulation via the electrodes to activate selected target muscles.

Additionally, or alternatively, electrical stimulation in the disclosed method may be applied on nerves or muscle-nerve junctions.

In some embodiments, the disclosed method further includes implanting at least one sensor in the patient's body. Optionally, the method includes synchronizing the electrical stimulation with at least one bodily signal acquired by the at least one sensor.

In one exemplary embodiment, the disclosed method is used to treat GERD by enhancing esophageal motility, thus enhancing motion of ingesta from the esophagus into the stomach.

In one exemplary embodiment, the disclosed method is used to treat GERD or gastroparesis by enhancing stomach motility, thus enhancing motion of ingesta from the stomach into the duodenum.

In one exemplary embodiment, the disclosed method is used to treat GERD by improving the contraction force of the lower esophageal sphincter (LES), thus reducing the backflow of ingesta from the stomach into the esophagus.

In one exemplary embodiment, the disclosed method is used to treat GERD by improving the contraction force of the upper esophageal sphincter (UES), thus reducing the backflow of ingesta from the esophagus into the pharynx.

In one exemplary embodiment, the disclosed method is used to treat fecal incontinence by improving the contraction force of the anal sphincters.

In one exemplary embodiment, the disclosed method is used to treat urinary incontinence by improving the contraction force of the urinary sphincters.

In one exemplary embodiment, the disclosed method is used to treat obesity or overweight by directing a backflow of ingesta from the stomach to the esophagus, causing unpleasant sensation, leading to decrease in appetite.

In one exemplary embodiment, the disclosed method is used to treat obesity or overweight by increasing internal pressure in the stomach, thus enhancing satiation feeling.

In embodiments where the disclosed method is used to treat obesity or overweight, electrical stimulation is applied before, during or after meals. Alternatively, stimulation is applied between meals.

In one exemplary embodiment, the disclosed method is used to treat constipation by enhancing bowel motility, thus enhancing flow of ingesta along the digestive tract toward the rectum.

In some embodiments, the disclosed method includes applying electrical stimulation in synchronization with motoric or sensory events associated with the digestive tract, such as meals, sensation of gas boluses, urge to defecate, or urge to urinate. Additionally, or alternatively, stimulation is applied at specific times, or continuously. Additionally, or alternatively, stimulation is applied at the discretion of the user, a physician, a machine, or any combination thereof.

An exemplary embodiment of the disclosed subject matter is a subcutaneously implantable device for treating patients suffering from digestive system disorders, the device comprising signal generator and a plurality of electrodes. Upon activation, the implantable device may generate electrical stimulation in order to treat various digestive symptoms or diseases.

In some embodiments, the disclosed device further comprises at least one sensor.

In some embodiments the sensor is an implantable sensor adapted to be implanted in the patient's esophageal wall and senses the esophageal acidity. The sensor is configured for outputting the acidity level to the processor via wired or wireless connection. In some embodiments the controller of the device receives the acidity monitoring data for controlling treatment parameters such as stimulation timing, stimulation intensity, stimulation waveform, stimulation frequency and the liker, for optimizing the therapeutic effect. In some embodiments the sensor is implanted for a temporary period.

In some embodiments, the disclosed device includes a biocompatible, hermetically sealed shell encapsulating components such as mechanical elements, electrical circuitry, processor or computing unit, power source, sensing elements and electrodes.

In other embodiments, the disclosed device includes one main shell and at least one additional shell, the main shell encapsulating a processor or computing unit, and the components encapsulated in the at least one additional shell are controlled by the main shell. Communication between the main shell and the at least one additional shell may be established by a wired connection, wireless connection, or a combination thereof.

In some embodiments, the shells are dimensioned such that they are suitable for subcutaneous implantation.

In some embodiments, a maximal longitudinal, transverse or depth dimension of the shell is less than 10 mm, 7 mm, 5 mm, 3 mm, 2 mm.

In some embodiments, the disclosed device includes a rechargeable battery. In some embodiments, the rechargeable battery is rechargeable wirelessly by means of a charger that is located outside the patient's body. In other embodiments, a charging port wired to the implantable device is disposed in a percutaneous manner, such that it is accessible from outside the patient's body. In other embodiments, mechanical energy exploited from bodily movements is utilized for charging the rechargeable battery without the need of an external charger. In other embodiments, the disclosed device includes a non-rechargeable battery.

In some embodiments, the disclosed device includes at least two electrodes. During device installation, the electrodes are implanted such that they are in electrical contact with at least one target region. Target regions may include muscles, nerves, muscle-nerve junction, or a combination thereof.

In some embodiments, the electrodes are designed as intramuscular, epimysial or nerve cuff electrodes. Additionally, or alternatively, the electrodes may be designed in any other way known in the art for delivering electrical stimulation to muscles, nerves or muscle-nerve junctions.

In some embodiments, the electrodes are further designed to acquire and deliver electrical signals from a target region to the device.

In some embodiments, the electrodes deliver electrical stimulation produced by the device's signal generator to target regions, such that electrical current flows from one electrode toward at least one other electrode through a portion of the patient's body.

In some embodiments, the electrical stimulation includes current-controlled or voltage-controlled pulses. Pulses may be monophasic, biphasic, or any combination thereof. Biphasic pulses may be asymmetrical or symmetrical with respect to a reference voltage or current. Pulses may have any shape known in the art for being utilized in electrical stimulation devices, such as rectangular pulses, triangular pulses, or sinusoidal pulses.

In some embodiments, stimulation pulses are characterized by maximal voltage of 10V, 20V, 30V, 40V (base to peak), and maximal current of 0.1 mA, 1 mA, 5 mA, 10 mA, 20 mA.

In some embodiments, stimulation pulses are cyclically repeated for a predetermined duration.

In some embodiments, the electrical stimulation includes bursts of repeated pulses. Stimulation bursts may be modulated such that they include substantially identical pulses, progressively increasing pulses, progressively decreasing pulses, or any other desired modulation method.

In some embodiments, pulse repetition frequency may be in the range of 10 Hz-200 Hz.

In some embodiments, pulse width may be in the range of 20 μs-1000 μs.

In some embodiments, stimulation bursts are characterized by burst repetition frequency in the range of 0.1 Hz-10 Hz.

In some embodiments, stimulation bursts may be symmetrical, asymmetrical, or any combination thereof, wherein a symmetrical burst is a current or voltage waveform in which a time of ramp up of the current or voltage is different from a time of ramp down.

In some embodiments, either of the disclosed device′ parameters may be finetuned to elicit an optimal treatment. Optimized parameters may include, among others, implantation location, electrode placement location, device activation times, and electrical stimulation characteristics. In some embodiments, an initial setup session may be required, upon the results of which optimized parameters may be determined.

In some embodiments, parameters characterizing the disclosed device construction and the electrical stimulation are set in accordance with the desired physiological effect. Among the parameters that can be set are the number and location of electrodes, electrode activation sequence, pulse control method (current control or voltage control), stimulation intensity, pulse shape, pulse frequency, burst shape, burst frequency, and stimulation timing.

In some embodiments, a plurality of electrodes is positioned in different locations in the abdominal musculature, and stimulation is applied through the electrodes in a sequential manner, such that abdominal muscle contraction is invoked in a desired sequence. In one exemplary embodiment, stimulation is applied via electrodes that are positioned at different heights on the rectus abdominis and external oblique muscles, such that abdominal musculature contracts in a sequential manner from the upper abdomen toward the lower abdomen, thus enhancing the propagation of digesta in the digestive tract in a desired direction toward the rectum.

In some embodiments, a shape of a stimulation burst is set in accordance with a desired physiological effect. In one exemplary embodiment, the electrical stimulation includes a series of controlled-current pulse bursts set in an asymmetrical shape, such that the burst ramp-up time is substantially shorter than the burst ramp-down time. Such stimulation can potentially enhance esophageal motility and relieve GERD symptoms. Potentially, the enhancement is mediated via a vibration-conveyor mechanism, where a structure carrying moveable substances is linearly displaced in a cyclic manner such that its motion in one direction is fast whereas its motion in the other direction is slower. Using this mechanism, the substances can have a net displacement in the general direction of the slow motion. Potentially, applying the shape of a stimulation burst can induce ingesta flow from the esophagus to the stomach by the mechanism.

In another exemplary embodiment, a similar stimulation is applied, however the burst ramp-up time is substantially longer than the burst ramp-down time. Such stimulation can potentially enhance backflow of ingesta from the stomach into the esophagus or induce nausea or vomiting.

In another exemplary embodiment, electrical stimulation is applied at the lower part of the abdominal musculature and includes a series of controlled-current pulse bursts set in an asymmetrical shape, such that the burst ramp-up time is substantially shorter than the burst ramp-down time. Such stimulation can potentially enhance motility of the lower parts of the digestive tract and propel digesta towards the rectum and anus.

In some embodiments, the construction and stimulation parameters of the disclosed device are optimized for improving the functionality of one or more of the digestive tract sphincters.

In one exemplary embodiment, electrical stimulation is applied to the rectus abdominis and external oblique muscles such as repetitive instances of rapid increase in intraabdominal pressure are induced. Rapidly increasing the intraabdominal pressure can potentially invoke natural bodily reflexes such as the straining esophageal reflex or the straining crural reflex, which in turn can increase the tonus of the LES or the UES. Alternatively, repetitive contractions of the LES can be induced in ways other than natural reflexes. Optionally, repeated activation of the LES and/or UES, mediated either the reflexes or otherwise, may lead to prolonged improvement in the competence of the sphincters. Optionally, contraction repetition frequency and intensity are set to induce a desired level of LES muscle loading and resting, thus optimizing LES strengthening. In another exemplary embodiment, similar stimulation is applied to the lower abdominal musculature, thus inducing repetitive contractions of the pelvic floor musculature, thereby strengthening the anal sphincter and/or urinary sphincter.

In some embodiments, the disclosed device includes at least one sensor adapted for acquiring bodily signals.

In some embodiments, the at least one sensor is based on either of the following: accelerometer, gyroscope, magnetic compass, inclinometer, piezoelectric sensor, electrocardiogram (ECG), electroencephalogram (EEG), electromyograph (EMG), microphone, electric impedance sensor, oximeter, optical interferometer, pH meter, and opto-sensor.

In some embodiments, the acquired at least one signal is an outcome of a physiological phenomenon which may influence the effectiveness of the electrical stimulation treatment. Such phenomena may include, among others, breathing, heart beating, blood pressure, muscular or ligamentous tension, body posture, arousal condition, or muscle activity level.

In some embodiments, the electrical stimulation is synchronized with one or more of the acquired signals.

A potential advantage of synchronizing electrical stimulation with physiological phenomena is eliciting the effectiveness of the electrical stimulation treatment. For example, electrically stimulating the abdominal musculature during breathing exhalation phase may lead to increased backflow of ingesta from the stomach to the esophagus, resulting from increased abdominal pressure while the contraction force of the LES is reduced due to relaxed diaphragm. On the other hand, electrically stimulating the abdominal musculature during inhalation phase may enhance the effectiveness of conveying the mechanical effect of the abdominal musculature contraction to the LES musculature, as a result of the increased abdominal tension and/or stiffness. Consequently, synchronizing the stimulation with the breathing cycle such that stimulation is active only during inhalation may elicit the treatment effectiveness if the stimulation is intended to reduce GERD symptoms. On the contrary, synchronizing the stimulation with the breathing cycle such that stimulation is active during exhalation may elicit the treatment effectiveness if the stimulation is intended to decrease appetite.

In some embodiments, the electrical stimulation is synchronized with the breathing cycle such that stimulation is active during inhalation phase. Alternatively, the electrical stimulation is synchronized with the breathing cycle such that stimulation is active during exhalation phase.

In some embodiments, the electrical stimulation is synchronized with intraabdominal pressure, such that stimulation is active while the intraabdominal pressure is maximized. Alternatively, the electrical stimulation is synchronized with intraabdominal pressure, such that stimulation is active while the intraabdominal pressure is minimized.

In some embodiments, the disclosed device is controllable by a user via wireless communication. The communication may be realized by any available wireless communication technology. Examples may include wi-fi, Bluetooth, Bluetooth low energy (BLE), or near field communication (NFC). Additionally, or alternatively, a custom communication protocol may be utilized.

In some embodiments, a smartphone may be used as a communication device used for controlling the implantable device. Alternatively, a different standalone remote-control device may be used.

One exemplary embodiment of the disclosed subject matter is a method for treating gastroesophageal reflux disease (GERD) in a patient, comprising implanting at least two intramuscular electrodes in the rectus abdominis and external oblique muscles, implanting a control unit in subcutaneous location with the at least two electrodes wired to the control unit by means of subcutaneous leads, and applying a series of electrical bursts to the electrodes wherein the electrical bursts are adapted for electrically stimulating the target muscles and/or nerves of the patient, thereby reducing backflow of ingesta from the stomach to the esophagus and relieving GERD symptoms.

According to some embodiments, the method further comprises applying a series of electrical asymmetrical bursts, thereby generating asymmetrical contractions of the target muscles.

In some embodiments, a direction of a fast phase of the bursts is upward and a direction of a slow phase of the bursts is downward. Potentially, such bursts are capable of inducing movements of the patient's digestive system.

In some embodiments, the movements are for causing the content of the esophagus to return to the stomach and relieving GERD symptoms.

According to some embodiments, the order of fast and slow phases of the burst is interchangeable, thereby changing the direction of induced motion of digesta in the digestive tract.

According to some embodiments, the stimulation bursts are symmetrical.

According to some embodiments, the method further comprises including a pulse generator in the control unit for generating the electrical stimulation.

According to some embodiments, the electrical stimulation is characterized by maximum voltage range being 10V, 20V, 30V, 40V (base to peak), maximum current range being 0.1 mA, 1 mA, 5 mA, 10 mA, 20 mA; pulse frequency range being 10 Hz-200 Hz, burst frequency range being 0.1 Hz-10 Hz, and pulse duration range being 20 μS-1000 μS.

According to some embodiments, the method further comprises including at least one sensor designed to acquire bodily signals.

According to some embodiments, the bodily signals comprise at least one member of a group consisting: breathing, heart pulse, ECG, blood pressure, stomach and esophagus pressure and position, monotony movements and body position.

According to some embodiments, the sensor is at least one member of a group consisting: piezoelectric sensor, accelerometer, ECG sensor, EEG sensor, EMG sensor, gyroscope, and opto-sensor.

According to some embodiments, the method further comprises synchronizing the stimulation with the bodily signals.

According to some embodiments, the synchronizing includes activating the electrical stimulation when the diaphragm is tensed. In other embodiments, the electrical stimulation is activated when the diaphragm is relaxed. Additionally, or alternatively, the synchronizing includes activating the electrical stimulation when the abdominal pressure is increased. In other embodiments, the electrical stimulation is activated when the abdominal pressure is decreased. Additionally, or alternatively, the synchronizing includes activating the electrical stimulation during breathing inhalation phase. In other embodiments, the electrical stimulation is activated during breathing exhalation phase.

According to some embodiments, the implanted device is wirelessly controllable by a user of the device. In some embodiments, the controlling includes activating and deactivating the electrical stimulation and setting the stimulation intensity. In some embodiments, the wireless control is utilized by means of a smartphone.

One other exemplary embodiment of the disclosed subject matter is a device for treating obesity; the device comprising a subcutaneously implanted electrodes and a control unit, the control unit further including a pulse generator for providing electrical stimulation signals to the electrodes, wherein the electrical stimulation bursts being adapted for inducing backflow of ingesta from the stomach into the esophagus. In some other embodiments, the electrical stimulation bursts are adapted for increasing the intraabdominal pressure, thereby enhancing satiation feeling.

One other exemplary embodiment of the disclosed subject matter is a device for treating constipation or paralysis of the stomach; the device comprises a subcutaneously implanted electrodes and a control unit, the control unit further including a pulse generator for providing electrical stimulation signals to the electrodes; wherein the electrical stimulation bursts being adapted for increasing stomach motility and enhancing propagation of digesta from the stomach into the duodenum. In some other embodiments, the electrical stimulation bursts are adapted for causing digesta in the transverse colon to propagate into the sigmoid colon.

One technical problem disclosed by the present disclosure is how to reduce the risk or complexity caused by deep implantation of electrodes to electrically stimulate internal muscles, such as the LES or crura.

One technical solution is to electrically stimulate external muscles such as the external oblique and rectus abdominis to invoke bodily reflexes that act on target internal muscles by implanting subcutaneous electrodes in area of the external muscles.

Before explaining at least one embodiment of the invention in detail, it is to be understood that the invention is not necessarily limited in its application to the details of construction and the arrangement of the components and/or methods set forth in the following description and/or stranded in the drawings. The invention is capable of other embodiments or of being practiced or carried out in various ways.

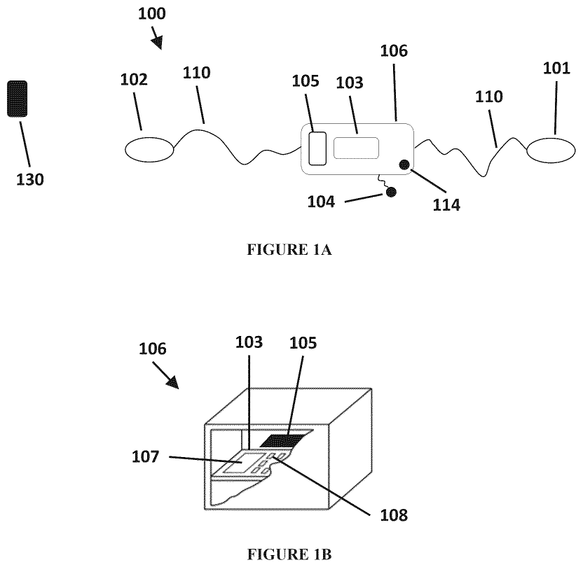

Reference is now made to FIG. 1A, showing a device 100 for treating digestive system disorders, in accordance with some embodiments of the disclosed subject matter. Device 100 includes a control unit 103 enclosed within a shell 106; plurality of electrodes 101, 102, and a sensor 104. Optionally, electrodes 101, 102 and sensor 104 are connected to control unit 103 via leads 110. In some embodiments, a sensor 114 is disposed within or on an external surface of shell 106. Optionally, electrodes 101, 102 are made from biocompatible, nonirritating material.

In some embodiments, device 100 is controllable by a remote-control device 130, using wireless communication.

Reference is now made to FIG. 1B, showing in more detail the construction of shell 106. In some embodiments, shell 106 is designed to house the electric circuitry and electrical components required for generating and controlling the electrical stimulation. Optionally, shell 106 is hermetically sealed to prevent ingress of bodily fluids or particles when implanted in a patient's body. Optionally, shell 106 is made of a flexible or semi-flexible material in order to allow good fit with the patient's body. Optionally, shell 106 is made of biocompatible material. Optionally, shell 106 is made of electrically insulating material.

In some embodiments, control unit 103 is disposed within shell 106. Optionally, shell 106 includes electrical circuitry board 108 and a power source such as a battery 105. Optionally, electrical circuitry board 108 includes pulse generator 107, as well as additional electrical components such as microcontroller, digital circuits, memory, communication, high voltage circuits, analog circuits, high voltages switches and bridges, protection circuits. In some embodiments, either of control unit 103, circuitry board 108, battery 105 and pulse generator 107 or some combination thereof is housed in a separate shell.

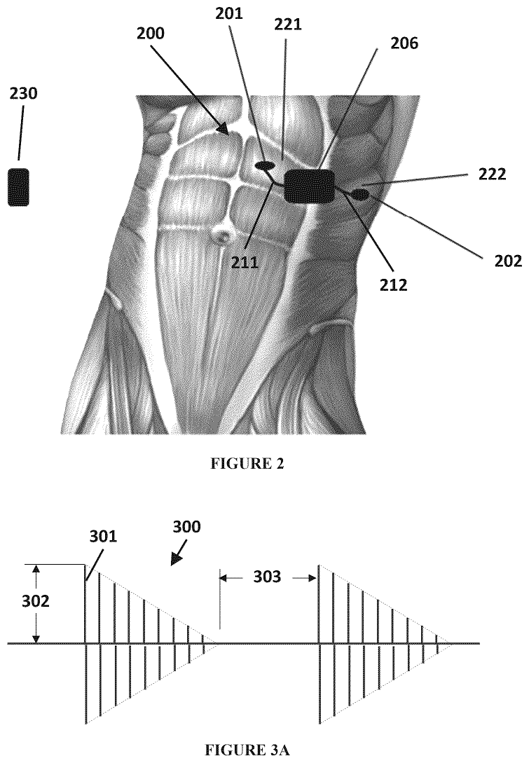

Reference is now made to FIG. 2, showing an exemplary embodiment of a device 200 for treating digestive system disorders, after being implanted in a patient's body. Device 200 includes shell 206 and two electrodes 201, 202. During the implantation procedure, electrodes 201 and 202 are positioned in electrical contact with the target regions rectus abdominus 221 and external oblique 222 muscles, respectively. Optionally, leads 211, 212 connect between the electrodes and shell 206.

Optionally, lead 211 is designed to have at least one electrical property dependent upon mechanical tension or elongation, for example tension-dependent electrical resistance or tension-dependent electrical capacitance. In such an embodiment, lead 211 serves as a mechanical tension or elongation sensor. Optionally, lead 211 is designed to provide electrical signal in correspondence with bodily movements associated with breathing. Optionally or alternatively, a breathing sensor is included in device 200 as a separate component (not shown).

When activated, device 200 generates electrical stimulation that is carried by leads 211, 212 and electrodes 201, 202 to the target regions, thereby causing selected abdominal muscles to contract and relax in a predetermined pattern. Optionally, the electrical stimulation generated by device 200 is controllable by remote control 230, via wireless communication. Optionally, remote control 230 is a smartphone.

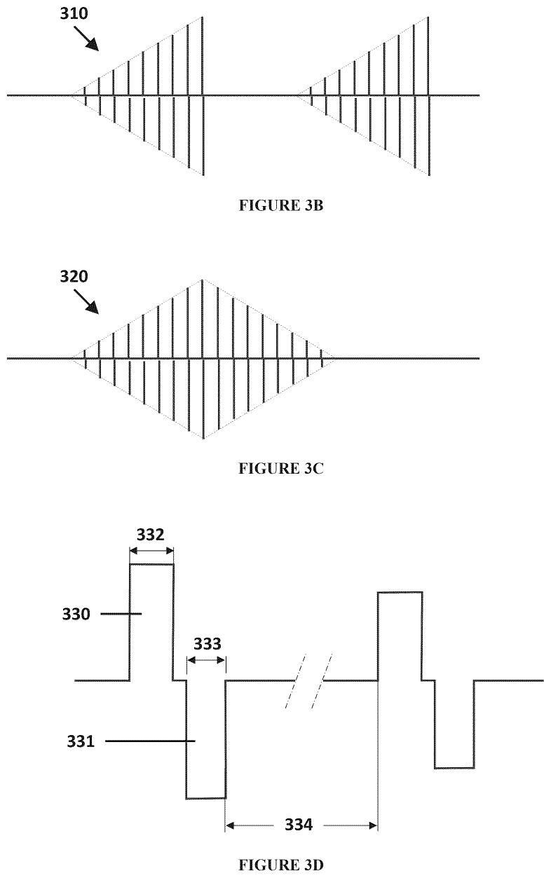

Reference is now made to FIGS. 3A-3B, showing waveforms of an exemplary electrical stimulation comprising asymmetrical bursts, wherein a direction of a fast phase of the bursts is upward and a direction of a slow phase of the bursts is downward, and to FIG. 3C, showing waveforms of an exemplary electrical stimulation comprising asymmetrical bursts, wherein a direction of a fast phase of the bursts is downward and a direction of a slow phase of the bursts is upward.

In FIG. 3A, a graphical representation of two instances of an exemplary asymmetrical burst 300 is shown, the burst containing a series of biphasic pulses in a sequentially declining order. The horizontal axis of the graphical representation represents time, whereas the vertical axis can represent electrical voltage or current. Optionally, the voltage or current of the burst's first pulse 301 corresponds to a desired intensity level 302. Optionally, intensity level 302 is adjustable by a user of the device. Optionally, the pulses' voltage or current linearly decreases from the burst first to last pulse. Alternatively, a difference burst form is applied. Optionally, an inter-burst duration 303 is kept between two consecutive bursts. Optionally, a user of the device. Alternatively, inter-burst duration 303 is adjustable by a machine.

In FIG. 3B, a graphical representation of two instances of an exemplary asymmetrical burst 310 is shown. Burst 310 is similar to burst 300, however the pulses' voltage or current linearly increases from the burst first to last pulse.

In FIG. 3C, a graphical representation of one instance of an exemplary symmetrical burst 320 is shown. Burst 320 is similar to bursts 300 and 310, however the pulses' voltage or current linearly increases and then decreases from the burst first to last pulse. Optionally, burst 320 is constructed by applying burst 310 followed by burst 300.

FIG. 3D shows a detailed view of two consecutive biphasic pulses of any one of bursts 300, 310, 320. Optionally, the pulses have rectangular shapes, representing constant current or constant voltage. Optionally, each pulse comprises a positive phase 330 followed by a negative phase 331. Alternatively, the order of positive and negative phases is reversed. Optionally, the pulse's phases are formed such that no net electrical charge is transferred during the pulse. Optionally, the positive phase duration 332 is identical to the negative phase duration 333. Optionally, inter-pulse duration 334 is kept between two consecutive pulses. Optionally, inter-pulse duration 334 is adjustable by a user of the device. Alternatively, inter-pulse duration 334 is adjustable by a machine.

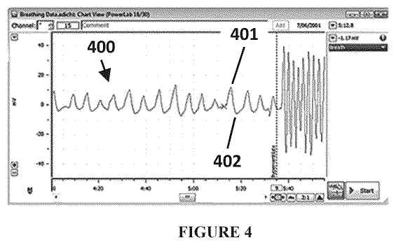

Reference is now made to FIG. 4, showing a portion of an exemplary signal 400 acquired by a breathing sensor that is included in an implantable device for treating digestive system disorders, in accordance with some embodiments of the disclosed subject matter. Optionally, signal 400 is a voltage signal output from a tension-dependent breathing sensor implanted in a patient's abdomen or thorax. Optionally, signal 400 is analyzable by a control unit included in the device, for example by identifying the signal's local maxima 401 and minima 402, so as to synchronize the electrical stimulation with the patient's breathing cycle.

1. A device for treating digestive system disorders, the device comprising:
a control unit;
a power source;
a pulse generator; and
a plurality of implanted electrodes comprising at least (i) a first electrode adapted to be positioned in electrical contact with a first abdominal muscle of a patient, and (ii) a second electrode adapted to be positioned in electrical contact with a second abdominal muscle of the patient;
wherein said pulse generator is adapted to deliver stimulation signals via said first and second electrodes to activate said first and second abdominal muscles, wherein said activating is adapted to induce instances of rapid increase in intraabdominal pressure in the patient which cause an increase in the tonus of the lower esophageal sphincter and/or the upper esophageal sphincter and/or invoke phasic contraction of the crural diaphragm, wherein said power source is adapted to provide power to said pulse generator, and wherein said control unit is adapted to control said pulse generator and said power source.

2. The device according to claim 1, wherein at least one of said control unit or signal generator is adapted to be implantable subcutaneously.

3. The device according to claim 1, wherein said digestive system disorders being at least one member of the group consisting of: gastroesophageal reflux disease (GERD), gastroparesis, obesity, incontinence, and constipation.

4. The device according to claim 1, wherein said electrical stimulation includes repetitive pulse bursts characterized by adjustable inter-burst duration.

5. The device according to claim 1, further comprising at least one sensor adapted to detect at least one of; intraabdominal pressure, breathing cycle, heartbeat, blood pressure, muscular or ligamentous tension, body posture, arousal condition, or muscle activity level.

6. The device according to claim 1, wherein said device comprises an implantable sensor adapted to be implanted the in the esophagus wall adapted-for sensing esophageal acidity level, and wherein said controller is further configured for controlling at least one parameter associated with said stimulation signals in accordance with said acidity level.

7. The device according to claim 1, wherein said first abdominal muscle is the rectus abdominus and said second abdominal muscle is the external oblique.

8. A method for treating digestive system disorders, the method comprising:
providing a device for treating digestive system disorders, the device comprising:
a control unit,
a power source,
a pulse generator, and
a plurality of implantable electrodes comprising at least (i) a first electrode adapted to be positioned in electrical contact with a first abdominal muscle of a patient, and (ii) a second electrode adapted to be positioned in electrical contact with a second abdominal muscle of the patient,
wherein said pulse generator is adapted to deliver stimulation signals via said first and second electrodes, wherein said power source is adapted to provide power to said pulse generator, and wherein said control unit is adapted to control said pulse generator and said power source;
implanting in said patient at least said first electrode in electrical contact with said first abdominal muscle and said second electrode in electrical contact with said second abdominal muscle; and
delivering said stimulation signals via said first and second electrodes to activate said first and second abdominal muscles, wherein said activating is adapted to induce instances of rapid increase in intraabdominal pressure in the patient which cause an increase in the tonus of the lower esophageal sphincter and/or the upper esophageal sphincter and/or invoke phasic contraction of the crural diaphragm.

9. The method according to claim 8, wherein said first abdominal muscle is the rectus abdominus and said second abdominal muscle is the external oblique.

10. (canceled)

11. The method according to claim 8, wherein at least one of said control unit or signal generator is adapted to be implantable subcutaneously, the method further comprising implanting at least one of said control unit or signal generator subcutaneously.

12. The method according to claim 8, wherein said digestive system disorders being at least one member of the group consisting of: gastroesophageal reflux disease (GERD), gastroparesis, obesity, incontinence, and constipation.

13. The method according to claim 8, wherein said increase in the tonus of the lower esophageal sphincter and/or the upper esophageal sphincter and/or phasic contraction of the crural diaphragm reduce the backflow of ingesta from the stomach into the esophagus or from the esophagus into the pharynx in the patient symptoms of said digestive system disorders.

14. (canceled)

15. The method according to claim 8, wherein said electrical stimulation includes repetitive pulse bursts characterized by adjustable inter-burst duration.

16. The method according to claim 8, wherein said device comprises at least one sensor adapted to detect at least one of:
intraabdominal pressure, breathing cycle, heartbeat, blood pressure, muscular or ligamentous tension, body posture, arousal condition, or muscle activity level.

17. The method according to claim 16, further comprising delivering said electrical stimulation in synchronization with said intraabdominal pressure.

18. The method according to claim 16, wherein said at least one sensor is selected from the group consisting of: accelerometer, gyroscope, magnetic compass, inclinometer, piezoelectric sensor, electrocardiogram (ECG), electroencephalogram (EEG), electromyograph (EMG), microphone, electric impedance sensor, oximeter, optical interferometer, pH meter, and opto-sensor.

19. (canceled)

20. (canceled)

21. The device according to claim 1, wherein said increase in the tonus of the lower esophageal sphincter and/or the upper esophageal sphincter and/or phasic contraction of the crural diaphragm reduce the backflow of ingesta from the stomach into the esophagus or from the esophagus into the pharynx in the patient, thereby alleviating symptoms of said digestive system disorders.

22. The device according to claim 5, wherein said at least one sensor is selected from the group consisting of: accelerometer, gyroscope, magnetic compass, inclinometer, piezoelectric sensor, electrocardiogram (ECG), electroencephalogram (EEG), electromyograph (EMG), microphone, electric impedance sensor, oximeter, optical interferometer, pH meter, and opto-sensor.

23. The device according to claim 5, wherein said intraabdominal pressure is determined based on an inhalation phase of said breathing cycle.

24. The device according to claim 5, wherein said electrical stimulation is delivered in synchronization with said intraabdominal pressure.
